(19) United States
(12) Patent Application Publication
(10) Pub. No.: US 2024/0182440 A1
(43) Pub. Date: Jun. 6, 2024

(54) PROCESS FOR PREPARING CHLORANTRANILIPROLE

(71) Applicant: Gharda Chemicals Limited, Thane, Maharashtra (IN)

(72) Inventors: Suchet Saran MATHUR, Thane, Maharashtra (IN); Hridaynath Vishwanath MHATRE, Thane, Maharashtra (IN); Vishal Parshuram PEDHAVI, Thane, Maharashtra (IN); Dinesh Krishna JAWALE, Thane, Maharashtra (IN); Uday Tukaram KOLI, Thane, Maharashtra (IN); Vaibhav Vijayshankar TRIPATHI, Thane, Maharashtra (IN)

(21) Appl. No.: 18/284,735

(22) PCT Filed: Aug. 9, 2021
(86) PCT No.: PCT/IB2021/056270

§ 371 Date: Sep. 28, 2023

(30) Foreign Application Priority Data
Apr. 2, 2021 (IN) 202121015761

(51) Int. Cl. C07D 401/04

(52) U.S. Cl. CPC C07D 401/04

(57) ABSTRACT

The present disclosure relates to a process for preparing Chlorantraniliprole. The process is simple, efficient and economical. The process of the present disclosure provides a comparatively higher yield of Chlorantraniliprole with greater purity.

### FIELD

The present disclosure relates to a process for preparing Chlorantraniliprole.

### BACKGROUND

The background information herein below relates to the present disclosure but is not necessarily prior art.

Chlorantraniliprole is an insecticide of the ryanoid class. Chlorantraniliprole belongs to a new class of selective insecticides featuring a novel mode of action to control a range of pests. The structural representation of Chlorantraniliprole is as given below:

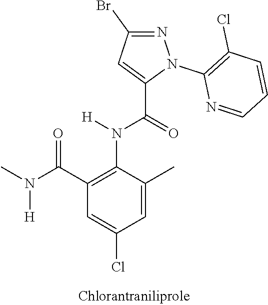 [SMILES: CNC(=O)c1cc(Cl)cc(C)c1NC(=O)c1cc(Br)nn1-c1ncccc1Cl]

Various methods for the preparation of Chlorantraniliprole are reported in the art. However, the product obtained by using the conventional methods uses pyridine or picoline as base which are costly & difficult to recover.

There is, therefore, felt a need to develop a process for preparing Chlorantraniliprole that mitigates the drawbacks mentioned hereinabove.

### OBJECTS

Some of the objects of the present disclosure, which at least one embodiment herein satisfies, are as follows:

An object of the present disclosure is to ameliorate one or more problems of the prior art or to at least provide a useful alternative.

Another object of the present disclosure is to provide a process for preparing Chlorantraniliprole.

Still another object of the present disclosure is to provide a process for preparing Chlorantraniliprole which is simple and economical.

Yet another object of the present disclosure is to provide a process for preparing Chlorantraniliprole with a comparatively high yield and high purity.

Yet another object of the present disclosure is to provide a process for preparing Chlorantraniliprole wherein solvents and reactants are recycled, thereby considerably reducing the effluent load and providing a green and environment friendly process.

Other objects and advantages of the present disclosure will be more apparent from the following description, which is not intended to limit the scope of the present disclosure.

### SUMMARY

The present disclosure relates to a process for preparing Chlorantraniliprole. The process comprises preparing an intermediate A (2-amino-5-chloro-3-methylbenzoic acid), an intermediate B (3-bromo-1-(3-chloropyridin-2-yl)-1H-pyrazole-5-carboxylic acid) and reacting the intermediate A and intermediate B to obtain Chlorantraniliprole. The intermediate A is prepared by firstly adding chloral hydrate, at least one alkali metal sulphate and hydroxylamine sulphate in a first fluid medium under stirring followed by addition of o-Toluidine and at least one first acid under stirring at a first predetermined temperature for a first predetermined time period to obtain 2-(hydroxyimino)-N-(2-tolyl)acetamide. 2-(hydroxyimino)-N-(2-tolyl)acetamide, which is then mixed with a second fluid medium followed by addition of at least one second acid under stirring at a second predetermined temperature for a second predetermined time period to obtain 7-methylisatin. 7-methylisatin is then mixed with at least one third acid and chlorinated by using a chlorinating agent optionally in the presence of ferric chloride and iodine under stirring at a third predetermined temperature for a third predetermined time period to obtain 5-chloro-7-methylisatin. 5-chloro-7-methylisatin is then mixed with at least one first aqueous base under stirring followed by addition of hydrogen peroxide under stirring at a fourth predetermined temperature for a fourth time period to the intermediate A (2-amino-5-chloro-3-methylbenzoic acid). Separately, an intermediate B (3-bromo-1-(3-chloropyridin-2-yl)-1H-pyrazole-5-carboxylic acid) is prepared by dissolving 2, 3-dichloropyridine in at least one third fluid medium under stirring followed by addition of at least one second base and hydrazine hydrate solution to obtain a mixture. The mixture is heated at a fifth predetermined temperature for a fifth predetermined time period to obtain 3-chloro-2-hydrazinopyridine. 3-chloro-2-hydrazinopyridine is reacted with an alkali metal alkoxide under stirring optionally in the presence of organo-metal complex as a catalyst, at a sixth predetermined temperature for a sixth predetermined time period followed by addition of di-isopropyl maleate for a seventh predetermined time period to obtain isopropyl 1-(3-chloropyridin-2-yl)-3-oxo-pyrazolidine-5-carboxylate. Isopropyl 1-(3-chloropyridin-2-yl)-3-oxo-pyrazolidine-5-carboxylate is then brominated by using at least one brominating agent in a fourth fluid medium under stirring at an eighth predetermined temperature for an eighth predetermined time period to obtain isopropyl 3-bromo-1-(3-chloropyridin-2-yl)-4,5-dihydro-1H-pyrazole-5-carboxylate.

Isopropyl 3-bromo-1-(3-chloropyridin-2-yl)-4,5-dihydro-1H-pyrazole-5-carboxylate is then mixed with at least one fifth fluid medium under stirring followed by addition of at least one alkali metal persulphate under stirring followed by addition of at least one fourth acid at a ninth predetermined temperature for a ninth predetermined time period to obtain isopropyl 3bromo-1-(3-chloro-2-pyridinyl)-1H-pyrazole-5-carboxylate. Isopropyl 3-bromo-1-(3-chloro -2-pyridinyl)-1H-pyrazole-5-carboxylate is then reacted with at least one third base in the presence of at least one phase transfer catalyst under stirring at a tenth predetermined temperature for a tenth predetermined time period to obtain the intermediate B (3-bromo-1-(3-chloropyridin-2-yl)-1H-pyrazole-5-carboxylic acid).

The intermediate A is reacted with the intermediate B in a sixth fluid medium by using a fourth base, methane-sulfonyl chloride, and methylamine to obtain Chlorantraniliprole.

### DETAILED DESCRIPTION

Embodiments are provided so as to thoroughly and fully convey the scope of the present disclosure to the person skilled in the art. Numerous details are set forth, relating to specific components, and methods, to provide a complete understanding of embodiments of the present disclosure. It will be apparent to the person skilled in the art that the details provided in the embodiments should not be construed to limit the scope of the present disclosure. In some embodiments, known processes or well-known apparatus or structures, and well-known techniques are not described in detail.

The terminology used, in the present disclosure, is only for the purpose of explaining a particular embodiment and such terminology shall not be considered to limit the scope of the present disclosure. As used in the present disclosure, the forms “a,” “an,” and “the” may be intended to include the plural forms as well, unless the context clearly suggests otherwise. The terms “comprises,” “comprising,” “including,” and “having,” are open ended transitional phrases and therefore specify the presence of stated features, integers, steps, operations, elements, modules, units and/or components, but do not forbid the presence or addition of one or more other features, integers, steps, operations, elements, components, and/or groups thereof. The particular order of steps disclosed in the method and process of the present disclosure are not to be construed as necessarily requiring their performance as described or illustrated. It is also to be understood that additional or alternative steps may be employed.

The terms first, second, third, etc., should not be construed to limit the scope of the present disclosure as the aforementioned terms may be only used to distinguish one element, component, region, layer or section from another component, region, layer or section. Terms such as first, second, third etc., when used herein do not imply a specific sequence or order unless clearly suggested by the present disclosure.

Chlorantraniliprole is an insecticide of the ryanoid class. Ryanodine is a naturally occurring insecticide isolated from Ryania speciosa.

Various methods for the preparation of Chlorantraniliprole are reported in the art. However, the product obtained by using the conventional methods has comparatively low yield and low purity.

The present disclosure relates to a process for preparing Chlorantraniliprole.

The process comprises preparing an intermediate A.

In a first step, chloral hydrate, at least one alkali metal sulphate and hydroxylamine sulphate are added and mixed in a first fluid medium under stirring followed by addition of o-Toluidine and at least one first acid under stirring at a first predetermined temperature for a first predetermined time period to obtain 2-(hydroxyimino)-N-(2-tolyl)acetamide.

In accordance with the embodiments of the present disclosure, the first fluid medium is water.

In accordance with the embodiments of the present disclosure, the alkali metal sulphate is selected from sodium sulphate, calcium sulphate, and potassium sulphate. In an exemplary embodiment, the alkali metal sulphate is sodium sulphate.

In accordance with the embodiments of the present disclosure, the first acid is selected from sulphuric acid and hydrochloric acid. In an exemplary embodiment, the first acid is sulphuric acid.

In accordance with the embodiments of the present disclosure, the first predetermined temperature is in the range of 45° C. to 100° C. In an exemplary embodiment, the first predetermined temperature is 60° C.

In accordance with the embodiments of the present disclosure, the first predetermined time period is in the range of 1 hour to 15 hours. In an exemplary embodiment, the first predetermined time period is 12 hours.

In an exemplary embodiment, the first fluid medium is water, the alkali metal sulphate is sodium sulphate, the first acid is sulphuric acid, the first predetermined temperature is 60° C. and the first predetermined time period is 12 hours. A reaction scheme of the exemplary embodiment for the first step for the preparation of the intermediate A is represented as:

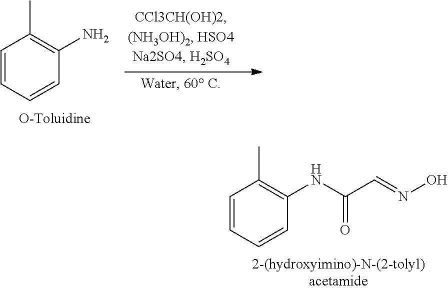

In a second step, 2-(hydroxyimino)-N-(2-tolyl)acetamide so obtained is mixed with a second fluid medium followed by addition of at least one second acid under stirring at a second predetermined temperature for a second predetermined time period to obtain 7-methylisatin.

In accordance with the embodiments of the present disclosure, the second fluid medium is selected from methylene dichloride, ethylene dichloride & chlorobenzene. In an exemplary embodiment, the second fluid medium is ethylene dichloride.

In accordance with the embodiments of the present disclosure, the second acid is selected from sulphuric acid, methanesulfonic acid, benzenesulfonic acid, Triflic acid and trifluoroacetic acid. In an exemplary embodiment, the second acid is sulphuric acid.

In accordance with the embodiments of the present disclosure, the second predetermined temperature is in the range of 25° C. to 40° C. In an exemplary embodiment, the second predetermined temperature is 28° C.

In accordance with the embodiments of the present disclosure, the second predetermined time period is in the range of 1 hour to 5 hours. In an exemplary embodiment, the second predetermined time period is 2.5 hours.

In an exemplary embodiment, the second fluid medium is ethylene dichloride, the second acid is sulphuric acid, the second predetermined temperature is 28° C. and the second predetermined time period is 2.5 hours. A reaction scheme for the exemplary embodiment of the second step for the preparation of the intermediate A is represented as:

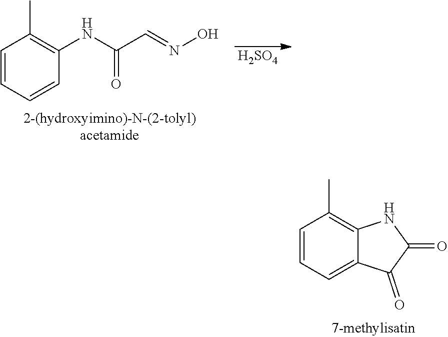

In a third step, 7-methylisatin is mixed with at least one third acid and chlorinated by using a chlorinating agent optionally in the presence of ferric chloride and iodine under stirring at a third predetermined temperature for a third predetermined time period to obtain 5-chloro-7-methylisatin.

In accordance with the embodiments of the present disclosure, the third acid is selected from hydrochloric acid, acetic acid and sulphuric acid. In an exemplary embodiment, the third acid is hydrochloric acid.

In accordance with an embodiment of the present disclosure, the normality of hydrochloric acid is in the range of 2N to 10N. In an exemplary embodiment, the normality of hydrochloric acid is 3N.

In accordance with the embodiments of the present disclosure, the chlorinating agent is chlorine gas.

In accordance with the embodiments of the present disclosure, the third predetermined temperature is in the range of 25° C. to 35° C. In an exemplary embodiment, the third predetermined temperature is 28° C.

In accordance with the embodiments of the present disclosure, the third predetermined time period is in the range of 1 hour to 16 hours. In an exemplary embodiment, the third predetermined time period is 9 hours.

In an exemplary embodiment, the third acid is hydrochloric acid having normality of 3N, the chlorinating agent is chlorine gas, the third predetermined temperature is 28° C., and the third predetermined time period is 9 hours. A reaction scheme for the exemplary embodiment of third step for the preparation of the intermediate A is represented as:

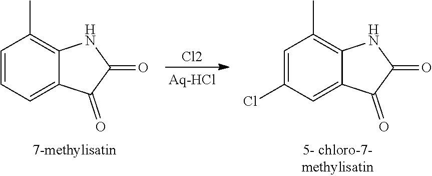

In a fourth step, 5-chloro-7-methylisatin is mixed with at least one first aqueous base under stirring followed by addition of hydrogen peroxide under stirring at a fourth predetermined temperature for a fourth time period to the intermediate A (2-amino-5-chloro-3-methylbenzoic acid).

In accordance with the embodiments of the present disclosure, a concentration of hydrogen peroxide is in the range of 10 to 30 wt. % in water.

In accordance with the embodiments of the present disclosure, the first base is selected from sodium hydroxide, potassium hydroxide, and ammonium hydroxide. In an exemplary embodiment, the first base is sodium hydroxide.

In accordance with the embodiments of the present disclosure, the normality of sodium hydroxide is in the range of IN to 10N. In an exemplary embodiment, the normality of sodium hydroxide is 2.6N.

In accordance with the embodiments of the present disclosure, a molar ratio of 5-chloro-7-methylisatin to the first base is in the range of 1:1.3 to 1:3.5. In an exemplary embodiment, the ratio is 1:2.1.

In accordance with the embodiments of the present disclosure, the fourth predetermined temperature is in the range of 50° C. to 80° C. In an exemplary embodiment, the fourth temperature is 60° C.

In accordance with the embodiments of the present disclosure, the fourth predetermined time period is in the range of 1 hour to 5 hours. In an exemplary embodiment, the fourth predetermined time period is 2.5 hours.

In an exemplary embodiment, a concentration of hydrogen peroxide is 30 wt. % in water, the first base is sodium hydroxide having normality of 2.6N, the fourth temperature is 60° C. and the fourth predetermined time period is 2.5 hours. A reaction scheme for the exemplary embodiment of the fourth step for the preparation of the intermediate A is represented as:

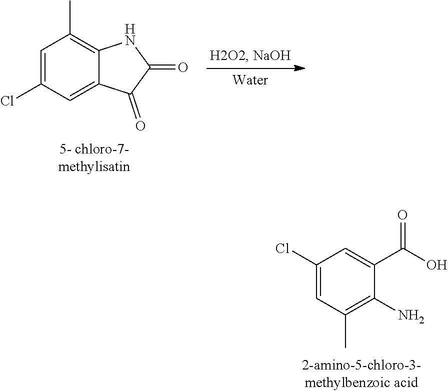

Separately, an intermediate B (3-bromo-1-(3-chloropyridin-2-yl)-1-H-pyrazole-5-carboxylic acid) is prepared.

In a first step for the preparation of the intermediate B, 2, 3-dichloropyridine is dissolved in at least one third fluid medium under stirring followed by addition of at least one second base and hydrazine hydrate solution to obtain a mixture. The mixture is heated at a fifth predetermined temperature for a fifth predetermined time period to obtain 3-chloro-2-hydrazinopyridine.

In an embodiment of the present disclosure, a concentration of the hydrazine hydrate is in the range of 75 to 80 wt. % in water.

In accordance with the present disclosure, a molar ratio of 2,3-dichloropyridine to the aqueous hydrazine is in the range of 1:1 to 1:6. In an exemplary embodiment of the present disclosure, the molar ratio of 2,3-dichloropyridine to hydrazine is 1:3. In accordance with the embodiments of the present disclosure, the third fluid medium is selected from the group consisting of diglyme (1-methoxy-2-(2-methoxyethoxy)ethane), monoglyme, 2-methoxyethanol, 2-ethoxyethanol, 2-butoxyethanol and n-butanol. In an exemplary embodiment, the third fluid medium is diglyme.

In accordance with the present disclosure, a molar ratio of 2,3-dichloropyridine to the third fluid medium is in the range of 1:2 to 1:4. In an exemplary embodiment of the present disclosure, the molar ratio of 2,3-dichloropyridine to diglyme is 1:3.

In accordance with the embodiments of the present disclosure, the second base is selected from the group consisting of potassium carbonate, sodium carbonate, sodium bicarbonate, potassium bicarbonate, sodium hydroxide, potassium hydroxide, magnesium carbonate, lithium carbonate, caesium carbonate and calcium carbonate. In an exemplary embodiment, the second base is sodium carbonate.

In accordance with the embodiments of the present disclosure, the fifth predetermined temperature is in the range of 90° C. to 125° C. In an exemplary embodiment, the fifth predetermined temperature is 105° C.

In accordance with the embodiments of the present disclosure, the fifth predetermined time period is in the range of 10 hours to 25 hours. In an exemplary embodiment of the present disclosure, the fifth predetermined time period is 22 hours.

In an exemplary embodiment, a concentration of the hydrazine hydrate is 78 wt. % in water, the third fluid medium is diglyme, the second base is sodium carbonate, the fifth predetermined temperature is 105° C., and the fifth predetermined time period is 22 hours. A reaction scheme for the exemplary embodiment of the first step for the preparation of the intermediate B is represented as:

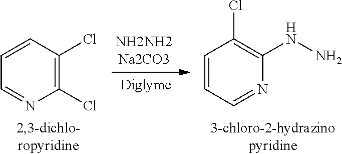 [SMILES: Clc1cccnc1Cl ; NNc1ncccc1Cl]

Separately di-isopropyl maleate is prepared using maleic anhydride and isopropyl alcohol in the presence of p-toluenesulfonic acid as catalyst in toluene as solvent. The product is distilled and used in further reaction.

In a second step, 3-chloro-2-hydrazinopyridine is reacted with an alkali metal alkoxide under stirring optionally in the presence of organo-metal complex as a catalyst, at a sixth predetermined temperature for a sixth predetermined time period followed by addition of di-isopropyl maleate for a seventh predetermined time period to obtain isopropyl 1-(3-chloropyridin-2-yl)-3-oxo-pyrazolidine-5-carboxylate.

In accordance with the embodiments of the present disclosure, the alkali metal alkoxide is prepared by reacting alkali metal with at least one anhydrous aliphatic alcohol having a carbon atom C1-C5.

In accordance with the embodiments of the present disclosure, at least one anhydrous aliphatic alcohol having carbon atom C1-C5 is selected from the group consisting of methanol, ethanol, n-propanol, isopropanol, n-butanol, and iso-butanol.

In an embodiment, the alkali metal is selected from the group consisting of lithium, sodium, potassium, and caesium. In an exemplary embodiment, the alkali metal is sodium.

In an exemplary embodiment, the alkali metal alkoxide is sodium isopropoxide.

In accordance with the embodiments of the present disclosure, the organo-metal complex is selected from an organo-palladium-complex and an organo-silver complex.

In accordance with the embodiments of the present disclosure, the organo-silver-complex is selected from the group consisting of Tris(triphenylphosphine)Silver(I) chloride, Tris(triphenylphosphine)Silver(I) bromide, Tris(triphenylphosphine)Silver(I) Iodide, Tris(triphenylphosphine)Silver(I) nitrate Bis(triphenylphosphine)Silver(I) chloride, Bis(triphenylphosphine)Silver(I) bromide, Bis(triphenylphosphine)Silver(I) Iodide, Bis(triphenylphosphine)Silver(I) nitrate Monotriphenyl phosphine monoiodosilver(I), Monotriphenyl phosphine monobromosilver(I), Monotriphenyl phosphine monochloro silver(I) and Monotriphenyl phosphine mononitro silver(I). In an exemplary embodiment, the organo-silver-complex is Tris(triphenylphosphine)Silver(I) chloride.

In accordance with the embodiments of the present disclosure, the organo-palladium-complex is selected from the group consisting of bis(triphenylphosphine)palladium (II) dichloride, bis(tricyclopentylphosphine)palladium (II) dichloride, bis(tricyclohexylphosphine)palladium (II) dichloride, bis(dibenzylideneacetone)palladium (0) and tetrakis(triphenylphosphine)palladium (0).

In accordance with the embodiments of the present disclosure, the sixth predetermined temperature and the seventh predetermined temperature is in the range of 5° C. to 35° C. In an embodiment, the sixth predetermined temperature and the seventh predetermined temperature is 10° C.

In accordance with the embodiments of the present disclosure, the sixth predetermined time period is in the range of 10 minutes to 30 minutes. In an exemplary embodiment, the sixth predetermined time period is 30 minutes.

In accordance with the embodiments of the present disclosure, the seventh predetermined time is in the range of 1.5 hours to 3.5 hours. In an exemplary embodiment, the seventh predetermined time is 2.5 hours.

In an exemplary embodiment, the alkali metal alkoxide is sodium isopropoxide, the organo-metal complex is organo-silver-complex is Tris(triphenylphosphine)Silver(I) iodide, the sixth predetermined temperature and the seventh predetermined temperature is 10° C., the sixth predetermined time period is 30 minutes, and the seventh predetermined time is 2.5 hours. A reaction scheme for the exemplary embodiment of the second step for the preparation of the intermediate B is represented as:

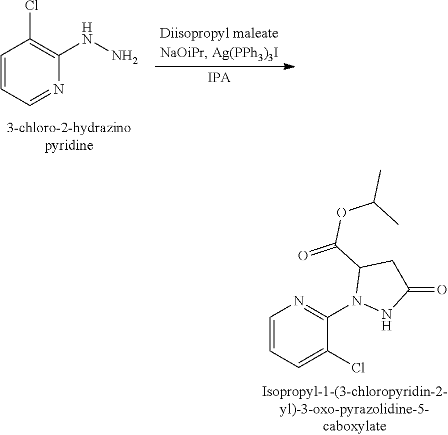 [SMILES: NNc1ncccc1Cl ; CC(C)OC(=O)C1CC(=O)NN1c1ncccc1Cl]

In a third step, Isopropyl 1-(3-chloropyridin-2-yl)-3-oxo-pyrazolidine-5-carboxylate is brominated by using at least one brominating agent in a fourth fluid medium under stirring at an eighth predetermined temperature for an eighth predetermined time period to obtain isopropyl 3-bromo-1-(3-chloropyridin-2-yl)-4,5 dihydro-1H-pyrazole-5-carboxylate.

In accordance with the embodiments of the present disclosure, the fourth fluid medium is chlorobenzene.

In accordance with the embodiments of the present disclosure, the brominating agent is selected from phosphoryl bromide (POBr₃), Phosphorus pentabromide (PBr₅), and Phosphorus tribromide (PBr₃).

In accordance with the embodiments of the present disclosure, the eighth predetermined temperature is in the range of 60° C. to 80° C. In an exemplary embodiment, the eighth predetermined temperature is 75° C.

In accordance with the embodiments of the present disclosure, the eighth predetermined time period is in the range of 1 hour to 4 hours. In an exemplary embodiment, the eighth predetermined time period is 2 hours.

In an exemplary embodiment, the fourth fluid medium is chlorobenzene, the brominating agent is 40 wt. % solution of phosphoryl bromide in acetonitrile, the eighth predetermined temperature is 75° C. and the eighth predetermined time period is 2 hours. A reaction scheme for the exemplary embodiment of the third step for the preparation of the intermediate B is represented as:

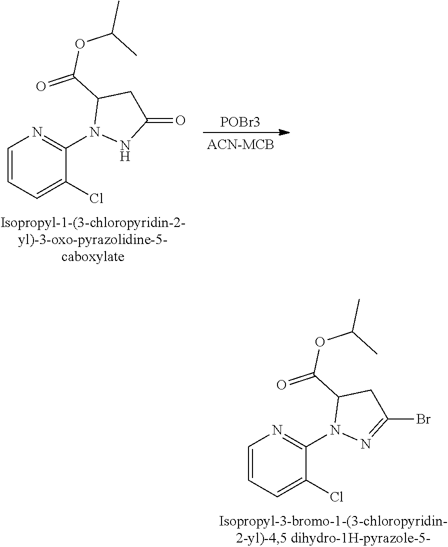

In a fourth step, Isopropyl 3-bromo-1-(3-chloropyridin-2-yl)-4,5-dihydro-1H-pyrazole-5-carboxylate is mixed with at least one fifth fluid medium under stirring followed by addition of at least one alkali metal persulphate under stirring followed by addition of at least one fourth acid at a ninth predetermined temperature for a ninth predetermined time period to obtain isopropyl 3-bromo-1-(3-chloro-2-pyridinyl)-1H-pyrazole-5-carboxylate.

In accordance with the embodiments of the present disclosure, the fifth fluid medium is at least one selected from acetonitrile and hexane. In an exemplary embodiment, the fifth fluid medium is a mixture of acetonitrile and hexane in a ratio of 90:10 (vol %).

In accordance with the embodiments of the present disclosure, the alkali metal persulphate is selected from sodium persulphate and potassium persulphate. In an exemplary embodiment, the alkali metal persulphate is potassium persulphate.

In accordance with the embodiments of the present disclosure, the fourth acid is selected from sulphuric acid and hydrochloric acid. In an exemplary embodiment, the fourth acid is sulphuric acid.

In accordance with the embodiments of the present disclosure, the ninth predetermined temperature is in the range of 60° C. to 90° C. In an exemplary embodiment, the ninth predetermined temperature is 65° C. and then raised to 80° C.

In accordance with the embodiments of the present disclosure, the ninth predetermined time period is in the range of 7 hours to 15 hours. In an exemplary embodiment, the predetermined time period is at least 8 hours.

In an exemplary embodiment, the fifth fluid medium is a mixture of acetonitrile and hexane in a ratio of 90:10 (vol %), the alkali metal persulphate is potassium persulphate, the fourth acid is sulphuric acid, the ninth predetermined temperature is 65° C. and then raised to 80° C. and, the predetermined time period is at least 8 hours. A reaction scheme for the exemplary embodiment of the fourth step for the preparation of the intermediate B is represented as:

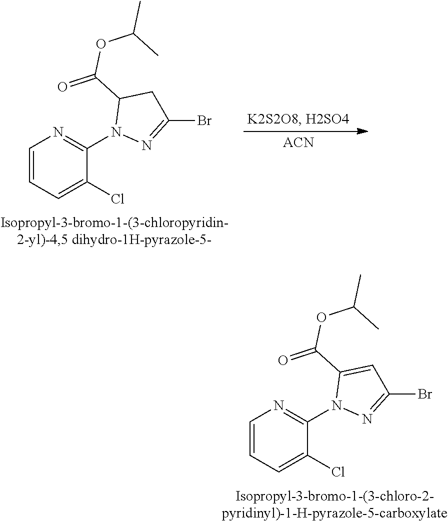 [SMILES: CC(C)OC(=O)C1CC(Br)=NN1c1ncccc1Cl ; "ACN" ; CC(C)OC(=O)c1cc(Br)nn1-c1ncccc1Cl]

In a fifth step, Isopropyl 3-bromo-1-(3-chloro-2-pyridinyl)-1H-pyrazole-5-carboxylate is reacted with at least one third base in the presence of at least one phase transfer catalyst under stirring at a tenth predetermined temperature for a tenth predetermined time period to obtain the intermediate B [3-bromo-1-(3-chloropyridin-2-yl)-1H-pyrazole-5-carboxylic acid].

In accordance with the embodiments of the present disclosure, the third base is selected from sodium hydroxide and potassium hydroxide. In an embodiment, the third base is sodium hydroxide having normality is the range of 3N to 10N. In an exemplary embodiment, the third base is sodium hydroxide having normality in the range of 5N.

In accordance with the embodiments of the present disclosure, the phase transfer catalyst is selected from tetra-n-butylammonium bromide and triethylbenzyl ammonium chloride.

In accordance with the embodiments of the present disclosure, the tenth predetermined temperature is in the range of 50° C. to 70° C. In an exemplary embodiment, the tenth predetermined temperature is 60° C.

In accordance with the embodiments of the present disclosure, the tenth predetermined time period is in the range of 1 hour to 12 hours. In an exemplary embodiment, the tenth predetermined time period is 6 hours.

In an exemplary embodiment, the third base is sodium hydroxide having normality of 5N, the phase transfer catalyst is tetra-n-butylammonium bromide, the tenth predetermined temperature is 60° C. and the tenth predetermined time period is 6 hours. A reaction scheme for the exemplary embodiment of the fifth step for the preparation of the intermediate B is represented as:

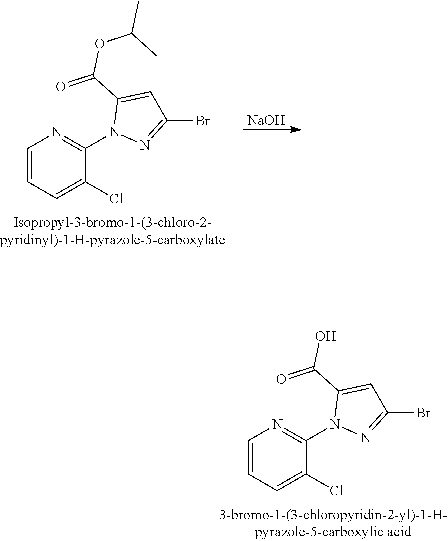

The intermediate A is reacted with the intermediate B in a sixth fluid medium by using a fourth base, methane-sulfonyl chloride and methylamine to obtain Chlorantraniliprole.

In an exemplary embodiment, intermediate B is first added to the sixth fluid medium followed by addition of the fourth base at a temperature in the range of 22 to 25° C. to obtain a thick slurry. Methane-sulfonyl chloride is then added to the thick slurry at a temperature in the range of 22 to 25° C. followed by stirring for 1 hour to obtain an equilibrated slurry. Intermediate A is added to the equilibrated slurry at temperature in the range of 22 to 25° C. followed by further addition of the fourth base and methane-sulfonyl chloride and equilibrating for 8 hours to obtain an equilibrated mixture. Methylamine is mixed with the equilibrated mixture to obtain Chlorantraniliprole.

The step of reacting intermediate A and intermediate B is carried out at ambient temperatures. Thus, the process of the present disclosure is energy efficient.

In accordance with the embodiments of the present disclosure, the sixth fluid medium is acetonitrile.

In accordance with the embodiments of the present disclosure, the fourth base is selected from sodium carbonate and potassium carbonate. In an exemplary embodiment, the fourth base is sodium carbonate. A reaction scheme for the embodiment of the reaction between intermediate A and intermediate B is represented as:

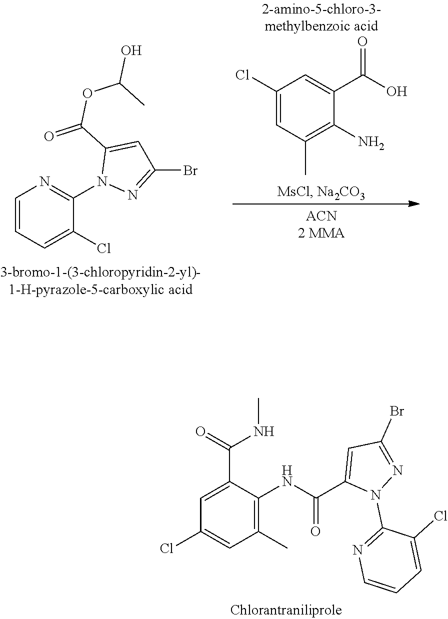 [SMILES: CC(O)OC(=O)c1cc(Br)nn1-c1ncccc1Cl ; Cc1cc(Cl)cc(C(=O)O)c1N ; CNC(=O)c1cc(Cl)cc(C)c1NC(=O)c1cc(Br)nn1-c1ncccc1Cl]

The process of the present disclosure employs inorganic bases which are cheaper than organic bases like pyridine, picolines, and the like; and therefore, the process of the present disclosure is cost-efficient and economical. The inorganic bases used in the process of the present disclosure can be easily separated.

In accordance with the embodiments of the present disclosure, the methanesulfonyl chloride is recovered and reused.

The present disclosure provides a simple process for the preparation of Chlorantraniliprole which provides a comparatively higher yield of the product with greater purity.

The foregoing description of the embodiments has been provided for purposes of illustration and not intended to limit the scope of the present disclosure. Individual components of a particular embodiment are generally not limited to that particular embodiment but are interchangeable. Such variations are not to be regarded as a departure from the present disclosure, and all such modifications are considered to be within the scope of the present disclosure.

The present disclosure is further illustrated herein below with the help of the following experiments. The experiments used herein are intended merely to facilitate an understanding of the ways in which the embodiments herein may be practiced and to further enable those of skill in the art to practice the embodiments herein. Accordingly, the experiments should not be construed as limiting the scope of embodiments herein. These laboratory scale experiments can be scaled up to industrial/commercial scale and the results obtained can be extrapolated to industrial/commercial scale.

### EXPERIMENTAL DETAILS

### Preparation of the Chlorantraniliprole as per the Process of Present Disclosure

### Experiment 1: Preparation of an intermediate A (2-amino-5-chloro-3-methylbenzoic acid):

a) Preparation of 2-(hydroxyimino)-N-(2-tolyl)acetamide: 8 litres of water was charged into the reactor followed by addition of 364 gm of chloral hydrate, 1.562 kgs of sodium sulphate and 522 gm of hydroxylamine sulphate under stirring at 28-32° C. to obtain a mixture. 218 gm of o-toluidine was then added under stirring to the mixture to obtain a turbid solution to which 86 gm of concentrated sulphuric acid was added under stirring for 30 minutes to obtain a reaction mass. The reaction mass was heated to 60-65° C. under stirring and maintained for 12 hrs. The reaction mass was a thin stirrable slurry.

Workup: The reaction was worked up and 470 gm of wet cake comprising 2-(hydroxyimino)-N-(2-tolyl) acetamide having 97% purity was obtained.

b) Preparation of 7-methylisatin: 940 gm of the wet cake from step a) (4 moles) was mixed and stirred with 1.2 litres of ethylene dichloride in a first reactor to form a slurry. In a second reactor 2.8 litres of 90% sulphuric acid was taken and the slurry from the first reactor was added at 28-32° C. for 2.5 hours to obtain a thick violet coloured stirrable mass. The violet coloured mass was monitored by HPLC and was taken for workup when the product from step a) (2-(hydroxyimino)-N-(2-tolyl)acetamide) was <1%.

Workup: The reaction mass was drowned in water and worked up; and 858 gm of wet cake comprising 7-methylisatin having 97% purity was obtained.

c) Preparation of 5-chloro-7-methylisatin: The wet cake from step b) cake was taken in 2.8 litres of 3N HCl to get stirrable slurry to which 8 gm of FeCl₃ was added along with 1 gm of iodine and then chlorine was passed through inlet in the stirrable slurry at 28-32° C. The reaction was monitored by using HPLC and reaction was stopped when step b) product (7-methylisatin) was <3% by HPLC.

Workup: The reaction mass was worked up and 885 gm of wet cake comprising 5-chloro-7-methylisatin having 96% purity was obtained.

### d) Preparation of 2-amino-5-chloro-3-methylbenzoic acid

In a reactor, 2.12 litres of 2.6N sodium hydroxide was taken and the wet cake of step c) (2.62 moles) was added at 28-32° C. under stirring for 30 minutes to obtain a dark green coloured thick slurry. The slurry was then heated to 60° C. under stirring and then 375 gm of 30% hydrogen peroxide was added in 2.5 hours maintaining temperature between 60-65° C. to obtain a clear dark solution. The reaction was monitored by using HPLC and was taken for workup when step c) product (5-chloro-7-methylisatin) was <0.5% by HPLC.

Workup: The reaction was worked up to obtain a wet cake which was dehydrated using chlorobenzene and 419 gm of dry cake comprising 2-amino-5-chloro-3-methylbenzoic acid having 95% purity was obtained.

### Experiment 2—Preparation of intermediate B (-3-bromo-1-(3-chloropyridin-2-yl)-1-H -pyrazole-5-carboxylic acid)

### Preparation of di-isopropyl maleate

1.5 kgs of maleic anhydride was mixed with 4.5 litres of toluene under stirring to obtain a thin stirrable slurry. 2.025 kgs of isopropanol (IPA) was added to the slurry along with 38 gm of p-toluenesulfonic acid under stirring to obtain a reaction mass. The reaction mass was heated to reflux and distilling water using water-toluene azeotrope to obtain a reaction mass. The volume of isopropanol which was co-distilled along with water was compensated. The reaction mass was taken for workup after no water was collected from reaction mass.

Workup: The reaction mass was worked up and 2.6 kgs of distilled material comprising di-isopropyl maleate was obtained.

### a) Preparation of 3-chloro-2-hydrazinopyridine

2 litres of diglyme was charged to the reactor under stirring and then 740 gm of 2,3-dichloropyridine was added to obtain a clear solution. 512 gm of sodium carbonate was added to the clear solution at 28-32° C. to obtain a thick stirrable slurry and stirred further for 30 minutes. Then, 960 gm of 78% aqueous hydrazine hydrate was added to the slurry and then heated to 105° C. under stirring to obtain a reaction mass. The reaction was monitoring by using HPLC and the reaction mass was taken for workup after it showed 2,3-dichloropyridine <2%.

Workup: The reaction mass was worked up and 674 gm of cake comprising 3-chloro-2-hydrazinopyridine having 99.8% purity was obtained.

b) Preparation of isopropyl 1-(3-chloropyridin-2-yl)-3-oxo-pyrazolidine-5-carboxylate: 3 litres of isopropanol (IPA) was charged into a reactor followed by addition of 53 gm of sodium metal to obtain a reaction mass. The reaction mass was heated under nitrogen atmosphere to reflux to obtain a clear solution. The clear solution was cooled to 10-15° C. and 293 gm of 3-chloro-2-hydrazinopyridine as prepared in step a) was added to obtain a thick stirrable slurry which was equilibrated at 10-15° C. for 30 mins. Then, 0.408 gm of Tris(triphenylphosphine)Silver(I) iodide was added to the slurry, equilibrated for 15 mins and to which 445 gm of di-isopropyl maleate was added at 10-15° C. under stirring for 2.5 hour to obtain a reaction mixture. Reaction was monitored using HPLC.

Workup: The reaction mixture was worked up to isolate unreacted 3-chloro-2-hydrazinopyridine (43 gm). The filtrate was worked up to isolate the product (isopropyl 1-(3-chloropyridin-2-yl)-3-oxo-pyrazolidine-5-carboxylate) as its HCl salt in 69% yield with 96% purity.

### Preparation of isopropyl 1-(3-chloropyridin-2-yl)-3-oxo-pyrazolidine-5-carboxylate in accordance with the present disclosure (without catalyst)

3 litres of isopropanol (IPA) was charged into a reactor followed by addition of 53 gm of sodium metal to obtain a reaction mass. The reaction mass was heated under nitrogen atmosphere to reflux to obtain a clear solution. The clear solution was cooled to 10-15° C. and 293 gm of 3-chloro-2-hydrazinopyridine was added to obtain a thick stirrable slurry which was equilibrated at 10-15° C. for 1 hour. 445 gm of di-isopropyl maleate was added to the slurry by maintaining the temperature of the slurry between 10-15° C. under stirring for 2.5 hours to obtain a reaction mixture. Reaction was monitored using HPLC.

Workup: The reaction mixture was worked up to isolate unreacted 3-chloro-2-hydrazinopyridine (47.6 gm) with 99% purity. The filtrate was worked up to isolate the product as its HCl salt in 65% yield with 96% purity.

### c) i) Preparation of isopropyl 3-bromo-1-(3-chloropyridin-2-yl)-4,5 dihydro-1H-pyrazole-5-carboxylate: (Using POBr3)

2.53 moles of the product as obtained in step b) of experiment 2 was mixed with the chlorobenzene to obtain a solution which was taken into a reactor and the solution was dehydrated. To the dehydrated solution, 1.2 kgs of POBr₃-Acetonitrile (40% wt.) solution was added to obtain a reaction mixture which was maintained at 70-75° C. for 2 hours. The so obtained reaction mixture was monitored by using HPLC and taken for workup after HPLC showed starting material <0.5%.

Workup: The reaction mass was worked up and 830 gm of cake comprising isopropyl 3-bromo-1-(3-chloropyridin-2-yl)-4,5-dihydro-1H-pyrazole-5-carboxylate having 98% purity was obtained.

### ii) Preparation of isopropyl 3-bromo-1-(3-chloropyridin-2-yl)-4,5 dihydro-1H-pyrazole-5-carboxylate: (Using PBr5)

In a reactor, 43 gms of phosphorus pentabromide was added to 300 ml of acetonitrile under stirring to get a stirrable slurry. 59 gms of step-b of experiment 2 product was added to the slurry under stirring in 20 minutes. The reaction mass was then heated to reflux and monitored by HPLC. The reaction was taken for workup after HPLC showed the starting material remained in the reaction mixture was <0.5%.

Workup: The reaction mass was worked up to get 64 gms of product with 98.6% purity.

### d) Preparation of isopropyl 3-bromo-1-(3-chloropyridin-2-yl)-1-H-pyrazole-5-carboxylate

702 gm of product from step c) was mixed with 2 litres of acetonitrile: hexane mixture (90:10 vol/vol) under stirring followed by addition of 772 gm of potassium persulphate to obtain a stirrable slurry. 40 gm of concentrated sulphuric acid was added to the slurry in 10 minutes followed by heating at 60-65° C. for 8 hours to obtain a reaction mass. After 8 hours, the reaction mass was concentrated at 78-82° C. and monitored using HPLC. The reaction mass was taken for workup after starting material was <0.5%.

Workup: The reaction mass was worked and an organic layer containing bromo-pyrazole ester was obtained.

e) Preparation of 3-bromo-1-(3-chloropyridin-2-yl)-1-H-pyrazole-5-carboxylic acid: 2.58 kgs of the filtrate from step d) (4.428 moles) was mixed with 1.1 litres of 5N NaOH and 0.71 gm of TBAB followed by heating to 60° C. under stirring to obtain a reaction mass. The reaction was monitored by using HPLC. The reaction was taken for workup when starting material <0.2%.

Workup: The reaction mass was worked up and 917 gm of cake was obtained which was purified to obtain 785 gm of 3-bromo-1-(3-chloropyridin-2-yl)-1-H-pyrazole-5-carboxylic acid having 96% purity.

### Experiment 3—Reaction of the Intermediate A with the Intermediate B to obtain Chlorantraniliprole

773 gm of intermediate B was mixed with 7.5 litres of acetonitrile to obtain a clear solution. 318 gm of sodium carbonate was added to the clear solution under stirring at 22-25° C. for 10 mins followed by equilibrating for 30 minutes to obtain a thick stirrable slurry. 322 gm of methanesulfonyl chloride was added to the thick stirrable slurry at 22-25° C. under stirring for 1 hour followed by adding 474 gm of intermediate A (prepared as per the present disclosure) under stirring for 15 mins to obtain a reaction mixture. The reaction mixture was equilibrated at 22-25° C. for 1 hour followed by addition of 318 gm of sodium carbonate and 321 gm of methanesulfonyl chloride at 22-25° C. for 4 hrs to obtain a reaction mass. Methylamine was bubbled through the reaction mass to obtain a final reaction mixture. The reaction was monitored by using HPLC. The reaction was terminated after HPLC showed the absence of intermediates.

Workup: The reaction mass was worked up and 1085 gm of cake comprising Chlorantraniliprole having 96% purity was obtained.

An aqueous layer as obtained after the work up of the reaction mass from experiment 3 (from 3.5 moles batch size) was concentrated to obtain 1.47 kg of an inorganic cake comprising sodium salt of methanesulfonic acid. The cake was mixed with ethylene dichloride in a concentration of 1.5 ml/gm of the cake along with a 2.28 mL of DMF/mole to obtain a slurry. Further, 1.1 kg of thionyl chloride was added below surface of the slurry at 50-55° C. for 7 hours. The reaction was monitored by gas evolution in scrubber.

Workup: The reaction mass was worked by filtration and distillation under vacuum to get 577 gms of methanesulfonyl chloride having 93% purity.

Methanesulfonyl chloride was recovered in 72% wt/wt after converting the sodium salt of MSA into its acid chloride using thionyl chloride in dichloroethane as solvent. Sodium salt was recovered from experiment 3 by concentrating aqueous layer completely.

### TECHNICAL ADVANCES AND ECONOMICAL SIGNIFICANCE

The present disclosure described herein above has several technical advantages including, but not limited to, the realization:
a simple, efficient and economical process for the preparation of Chlorantraniliprole; and
provides a comparatively high yield with higher purity of Chlorantraniliprole;
Methanesulfonyl chloride can be recovered and reused.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary. While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

1. A process for preparing Chlorantraniliprole, wherein said process comprising the following steps:
i. preparing an intermediate A (2-amino-5-chloro-3-methylbenzoic acid) by:
a) adding chloral hydrate, at least one alkali metal sulphate and hydroxylamine sulphate in a first fluid medium under stirring followed by adding o-Toluidine and at least one first acid under stirring at a first predetermined temperature for a first predetermined time period to obtain 2-(hydroxyimino)-N-(2-tolyl)acetamide;
b) mixing 2-(hydroxyimino)-N-(2-tolyl)acetamide in a second fluid medium followed by adding at least one second acid under stirring at a second predetermined temperature for a second predetermined time period to obtain 7-methylisatin;
c) mixing 7-methylisatin with at least one third acid and chlorinating 7-methylisatin by using a chlorinating agent optionally in the presence of ferric chloride and iodine under stirring at a third predetermined temperature for a third predetermined time period to obtain 5-chloro-7-methylisatin; and
d) mixing 5-chloro-7-methylisatin with at least one first aqueous base under stirring followed by adding hydrogen peroxide under stirring at a fourth predetermined temperature for a fourth time period to the intermediate A (2-amino-5-chloro-3-methylbenzoic acid),
ii. Separately preparing an intermediate B (3-bromo-1-(3-chloropyridin-2-yl)-1-H -pyrazole-5-carboxylic acid)
a) dissolving 2, 3-dichloropyridine in at least one third fluid medium under stirring followed by adding at least one second base and hydrazine hydrate solution to obtain a mixture and heating the mixture at a fifth predetermined temperature for a fifth predetermined time period to obtain 3-chloro-2-hydrazinopyridine;
b) reacting 3-chloro-2-hydrazinopyridine with an alkali metal alkoxide under stirring optionally in the presence of organo-metal complex as a catalyst, at a sixth predetermined temperature for a sixth predetermined time period followed by adding di-isopropyl maleate for a seventh predetermined time period to obtain isopropyl 1-(3-chloropyridin-2-yl)-3-oxo-pyrazolidine-5-carboxylate;
c) brominating isopropyl-1-(3-chloropyridin-2-yl)-3-oxo-pyrazolidine-5-carboxylate by using at least one brominating agent in a fourth fluid medium under stirring at an eighth predetermined temperature for an eighth predetermined time period to obtain isopropyl 3-bromo-1-(3-chloropyridin-2-yl)-4,5 dihydro-1H-pyrazole-5-carboxylate;
d) mixing isopropyl 3-bromo-1-(3-chloropyridin-2-yl)-4,5 dihydro-1H-pyrazole -5-carboxylate with at least one fifth fluid medium under stirring followed by adding at least one alkali metal persulphate under stirring followed by addition of at least one fourth acid at a ninth predetermined temperature for a ninth predetermined time period to obtain isopropyl 3-bromo-1-(3-chloro-2-pyridinyl)-1-H-pyrazole-5-carboxylate;
e) mixing isopropyl 3-bromo-1-(3-chloro-2-pyridinyl)-1-H-pyrazole-5-carboxylate with at least one third base and at least one phase transfer catalyst under stirring at a tenth predetermined temperature for a tenth predetermined time period to obtain the intermediate B (3-bromo-1-(3-chloropyridin-2-yl)-1-H-pyrazole-5-carboxylic acid); and
iii. reacting the intermediate A with the intermediate B in a sixth fluid medium by using a fourth base, methanesulfonyl chloride and methyl amine to obtain Chlorantraniliprole.

2. The process as claimed in claim 1, wherein the first fluid medium is water; the alkali metal sulphate is selected from sodium sulphate, calcium sulphate, and potassium sulphate; the first acid is selected from sulphuric acid and hydrochloric acid; the first predetermined temperature is in the range of 45° C. to 100° C.; and the first predetermined time period is in the range of 1 hour to 15 hours.

3. The process as claimed in claim 1, wherein the second fluid medium is selected from methylene dichloride and ethylene dichloride; the second acid is selected from sulphuric acid, methanesulfonic acid, benzenesulfonic acid, Triflic acid and trifluoroacetic acid; the second predetermined temperature is in the range of 25° C. to 40° C.; and the second predetermined time period is in the range of 1 hour to 5 hours.

4. The process as claimed in claim 1, wherein the third acid is selected from hydrochloric acid, acetic acid and sulphuric acid; the chlorinating agent is chlorine gas; the third predetermined temperature is in the range of 25° C. to 35° C.; and the third predetermined time period is in the range of 1 hour to 6 hours.

5. The process as claimed in claim 1, wherein a concentration of hydrogen peroxide is in the range of 10 to 30 wt. % in water; the first base is selected from sodium hydroxide, potassium hydroxide, and ammonium hydroxide; the fourth predetermined temperature is in the range of 50° ° C. to 80° C.; and the fourth predetermined time period is in the range of 1 hour to 5 hours.

6. The process as claimed in claim 1, wherein a concentration of the hydrazine hydrate is in the range of 75 to 80 wt. % in water; the third fluid medium is selected from the group consisting of diglyme (1-methoxy-2-(2-methoxyethoxy)ethane), monoglyme, 2-methoxyethanol, 2-ethoxyethanol, 2-butoxyethanol and n-butanol; the second base is selected from the group consisting of potassium carbonate, sodium carbonate, sodium bicarbonate, potassium bicarbonate, sodium hydroxide, potassium hydroxide, magnesium carbonate, lithium carbonate, caesium carbonate and calcium carbonate; the fifth predetermined temperature is in the range of 90° C. to 125° C.; and the fifth predetermined time period is in the range of 10 hours to 25 hours.

7. The process as claimed in claim 1, wherein the alkali metal alkoxide is prepared by reacted with at least one anhydrous aliphatic alcohol having a carbon atom C1-C5; wherein at least one anhydrous aliphatic alcohol having carbon atom C1-C5 is selected from the group consisting of methanol, ethanol, n-propanol, isopropanol, n-butanol, and iso-butanol; and; wherein the alkali metal is selected from the group consisting of lithium, sodium, potassium, and caesium.

8. The process as claimed in claim 1, wherein the organo-metal complex is selected from an organo-palladium-complex and an organo-silver complex; the sixth predetermined temperature and the seventh predetermined temperature is in the range of 5° C. to 35° C.; the sixth predetermined time period is in the range of 10 minutes to 30 minutes; and the seventh predetermined time is in the range of 1.5 hours to 3.5 hours.

9. The process as claimed in claim 8, wherein the organo-palladium-complex is selected from the group consisting of bis(triphenylphosphine)palladium (II) dichloride, bis(tricyclopentylphosphine)palladium (II) dichloride, bis(tricyclohexylphosphine)palladium (II) dichloride, bis(dibenzylideneacetone)palladium (0) and tetrakis(triphenylphosphine)palladium (0).

10. The process as claimed in claim 8, wherein the organo-silver-complex is selected from the group consisting of Tris(triphenylphosphine)Silver(I) chloride, Tris(triphenylphosphine)Silver(I) bromide, Tris(triphenylphosphine)Silver(I) Iodide, Tris(triphenylphosphine)Silver(I) nitrate Bis(triphenylphosphine)Silver(I) chloride, Bis(triphenylphosphine)Silver(I) bromide, Bis(triphenylphosphine)Silver(I) Iodide, Bis(triphenylphosphine)Silver(I) nitrate Monotriphenyl phosphine monoiodosilver(I), Monotriphenyl phosphine monobromosilver(I), Monotriphenyl phosphine monochloro silver(I) and Monotriphenyl phosphine mononitro silver(I).

11. The process as claimed in claim 1, wherein the fourth fluid medium is chlorobenzene; the brominating agent is selected from phosphoryl bromide, phosphorus pentabromide, and phosphorus tribromide; the eighth predetermined temperature is in the range of 60° C. to 80° C.; and the eighth predetermined time period is in the range of 1 hour to 4 hours.

12. The process as claimed in claim 1, wherein the fifth fluid medium is at least one selected from acetonitrile and hexane; the alkali metal persulphate is selected from sodium persulphate and potassium persulphate; the fourth acid is selected from sulphuric acid and hydrochloric acid; the ninth predetermined temperature is in the range of 60° C. to 90° C.; and the ninth predetermined time period is in the range of 7 hours to 15 hours.

13. The process as claimed in claim 1, wherein the third base is selected from sodium hydroxide and potassium hydroxide; the phase transfer catalyst is selected from tetra-n-butylammonium bromide and triethylbenzyl ammonium chloride; the tenth predetermined temperature is in the range of 50° C. to 70° C.; and the tenth predetermined time period is in the range of 1 hour to 12 hours.

14. The process as claimed in claim 1, wherein the sixth fluid medium is acetonitrile, and the fourth base is selected from sodium carbonate and potassium carbonate.

15. The process as claimed in claim 1, wherein the methanesulfonyl chloride is recovered and reused.
